(19) United States
(12) Patent Application Publication
(10) Pub. No.: US 2008/0234226 A1
(43) Pub. Date: Sep. 25, 2008

(54) Use Of Iron(III) Complex Compounds For The Preparation Of A Medicament For Oral Treatment Of Iron Deficiency States In Patients With Chronic Inflammatory Bowel Disease

(75) Inventors: Kari Erichsen, Bergen (NO); Bo Danielson, Hergiswil (CH)

Correspondence Address: RANKIN, HILL & CLARK LLP, 925 EUCLID AVENUE, SUITE 700, CLEVELAND, OH, 44115-1405, US

(73) Assignee: VIFOR (INTERNATIONAL) AG, St. Gallen (CH)

(21) Appl. No.: 12/064,053

(22) PCT Filed: Aug. 22, 2006
(86) PCT No.: PCT/EP2006/065532

§ 371 Date: Apr. 21, 2008

(30) Foreign Application Priority Data
Aug. 25, 2005 (EP) 05107790.7

(51) Int. Cl. A61K 31/721; A61P 1/00

(52) U.S. Cl. 514/59

(57) ABSTRACT

The use of iron(III) complex compounds with carbohydrates or derivatives thereof for the preparation of a medicament for oral treatment of iron deficiency states in patients with chronic inflammatory bowel disease, in particular Crohn's disease and colitis ulcerosa, is disclosed.

The present invention relates to novel therapeutic uses of iron(III) complex compounds with carbohydrates or derivatives thereof, in particular with dextrins or oxidation products of dextrins, namely for the preparation of medicaments for treatment of iron deficiency states in patients with chronic inflammatory bowel diseases, in particular Crohn's disease and/or colitis ulcerosa.

Iron deficiency is the most frequent trace element deficiency worldwide. Approx. 2 billion people worldwide suffer from iron deficiency or iron deficiency anaemia (E. M. DeMaeyer, “Preventing and controlling iron deficiency anaemia through primary health care”, World Health Organization, Geneva, 1989, ISBN 92 4 154249 7).

WO 95/35113 discloses the use of iron(III) oxide as an active compound for treatment of immunoinsuficiency diseases, in particular AIDS.

DE 1467980 discloses therapeutically usable iron injection preparations and processes for their preparation.

U.S. Pat. No. 3,076,798 discloses processes for the preparation of iron(III)-polymaltose complex compounds which are suitable for parenteral administration.

WO 04/037865 discloses the use of iron-carbohydrate complexes for treatment or prophylaxis of iron deficiency states.

WO 03/087164 discloses iron complex compounds with hydrogenated dextrins for treatment or prophylaxis of iron deficiency states.

WO 02/46241 discloses iron(III)-pullulan complex compounds and their use for treatment or prophylaxis of iron deficiency states.

WO 99/48533 discloses iron-dextran compounds for treatment of iron deficiency anaemia, which comprise hydrogenated dextran having a particular molecular weight of approx. 1,000 Dalton.

I. Maslovski, American Journal of Hematology, April 2005, vol. 78, no. 4, p. 261-264 discloses the activity of Ferrlecit®, an iron(III)-gluconate complex in sucrose having a molecular weight of 350,000, or Venofer®, an iron(III)-sucrose complex, for intravenous treatment of anaemic patients suffering from chronic inflammatory bowel disease.

G. Bodemar et al., Scandinavian Journal of Gastroenterology, May 2004, vol. 39, p. 454-458 describes iron(III)-sucrose compounds for intravenous treatment of anaemia in patients with Crohn's disease and ulcerative colitis.

DE-A-102 49 552 describes iron(III) complex compounds with maltodextrins and the (particularly preferably parenteral) use thereof for treatment of anaemia.

CH-A-694 197 describes iron(III)-polymaltose compounds for treatment of anaemia, but without giving indications of actions in the gastrointestinal tract or on IBD or Crohn's disease.

Iron sulfate is known to cause relatively frequently unpleasant dose-dependent side reactions, such as gastrointestinal disorders or a discoloration of the teeth. Iron from iron salt compounds is subject to passive diffusion of free iron ions. The iron can enter the circulation and as a result cause side reactions or an iron poisoning. Accordingly, the LD50 value in white mice of 230 mg iron/kg is relatively low.

The use of iron-dextran is disclosed in Oski et al. “Effect of Iron Therapy on Behavior Performance in Nonanemic, Iron-Deficient Infants”, PEDIATRICS 1983; volume 71; 877-880. Parenteral use of iron-dextran is disadvantageous because a dextran-induced anaphylactic shock may occur.

Inflammatory bowel diseases (IBD) include a group of diseases of the gastrointestinal tract which are characterized by intestinal inflammation and a chronic course with constant relapses. IBD has traditionally been characterized either as colitis ulcerosa or as Crohn's disease, based on clinical, radiological, endoscopic and histological criteria. Although the aetiology of IBD still requires definition, recent clinical and experimental studies suggest that the trigger and the pathogenesis of these diseases are multifactorial, and that interactions between genetic, environmental and immune factors are involved.

Inflammatory bowel diseases are not spread uniformly throughout the world. There is a clear tendency towards an increased occurrence in developed countries compared with less developed countries. The occurrence of IBD in Europe is approx. 390 cases per 100,000 people. Extrapolation of these figures to the European population of approx. 580 million gives an estimated number of 2.2 million people affected by IBD (Loftus E V, Jr., Gastroenterology 2004, 126, 11504-1517). Colitis ulcerosa and Crohn's disease are diagnosed most frequently in older adolescents and young adults, but can occur at any age.

Colitis ulcerosa is a disease of the mucous membrane which conventionally affects the rectum and then extends into the adjacent areas, so that all or part of the colon is affected. The spread is continuous, without areas of unaffected mucous membrane remaining. The main symptoms of colitis ulcerosa are violent diarrhea, rectal bleeding, mucous discharge and cramp-like abdominal pain. The severity of the symptoms correlates with the extent of the disease.

Crohn's disease can affect any region of the gastrointestinal tract from the mouth to the anus, but most frequently relates to the small intestine and/or the colon. The inflammation is transmural and segmental, normal areas existing between the areas of diseased intestine. Consequences of the inflammation include fistulation on other loops of the intestine, the urinary bladder, the vagina or the perianal skin, abdominal or perianal abscesses and narrowing of the intestine. The location and the course of the disease influence the clinical manifestations. The most frequent symptoms are diarrhea, cramp-like abdominal pain, fever, anorexia and weight loss.

Extraintestinal manifestations of colitis ulcerosa and Crohn's disease can relate to multiple organ systems, such as eyes, skin and joints, and equally gastrointestinal organs, including the liver and gallbladder.

Treatment comprises administration of anti-inflammatory agents and under certain circumstances antibiotics, and a change in diet. An operation may occasionally be necessary. Psychotherapy is furthermore often undertaken, on the one hand for the management of stress, which is also a triggering factor, and on the other hand for treatment of depression, which often arises as a consequence of the chronic ever-recurring symptoms (see e.g. Pschyrembel, Klinisches Wörterbuch, 256th edition, de Gruyter, p. 302/303, p. 443; http://familydoctor.org or http://www.mayoclinic.com).

Iron deficiency often occurs as a complication in patients with chronic inflammatory bowel disease. Chronic intestinal bleeding can lead to more iron being lost than is taken in through food. Conventional oral iron preparations, in general iron(II) salts, often cause severe gastrointestinal side effects, which leads to a poor patient compliance. Oral iron therapy can intensify the lesions of the intestinal tissue by catalysis of the formation of reactive oxygen species. Since free iron is a potent catalyst of the formation of reactive oxygen species, oral iron(II) therapy can even be harmful for patients with chronic inflammatory bowel disease. Oral iron(II) preparations are poorly absorbed and lead to high faecal iron concentrations, and a significant content of the faecal iron is available for the catalytic activity. If iron comes into contact with the inflamed intestinal mucosa, it can increase the production of reactive oxygen species and as a result intensify tissue damage. It is therefore particularly important for patients with chronic inflammatory bowel disease to have available readily tolerated iron preparations.

Iron(III)-polymaltose complex contains iron in a nonionic form, which is less toxic. Fewer side effects occur on administration of compounds of this type, and patient compliance is improved compared with iron(II) sulfate (Jacobs, P., Wood, L., Bird, A R., Hematol. 2000, 5:77-83). However, there is not yet any experience or reports of the use of iron(III)-polymaltose complex in patients with chronic inflammatory bowel disease.

The inventors therefore had the object of discovering readily tolerated iron compounds which are suitable for treatment of iron deficiency states in patients with chronic inflammatory bowel disease.

They were able to demonstrate in a study that iron(III) complex compounds with carbohydrates, in particular with polymaltose (maltodextrin), are tolerated in particular and have a high patient compliance. In this study, it was surprising that under treatment with the iron(III) complexes no oxidative stress occurred, in contrast to treatment with iron(II) sulfate, under which a significant increase in plasma malondialdehyde (MDA), a lipid peroxidation marker, was observed.

Oxidative stress, in particular lipid peroxidation, is associated with an increased risk of suffering from cardiac infarction, cancer and atherosclerosis. Oxidative modification of low-density lipoprotein (LDL) is held responsible for atherogenesis (see references given in Tuomainen et al., Nutrition Research, vol. 19, no. 8, pp. 1121-1132, 1999).

Iron(III)-polymaltose complex compounds indeed lead to only a slow increase in the ferritin level, but are used more efficiently for haemoglobin synthesis (T.-P. Tuomainen et al., loc. cit., p. 1127). The inventors have provided the present invention on the basis of these results.

The present invention therefore provides the use of iron(III) complex compounds with carbohydrates or derivatives thereof for the preparation of a medicament for treatment of iron deficiency states in patients with chronic inflammatory bowel disease.

According to the invention, iron deficiency state is understood as meaning a state in which haemoglobin, iron and ferritin levels in the plasma are reduced and transferrin is increased, which leads to a reduced transferrin saturation.

The state to be treated according to the invention includes iron deficiency anaemia and iron deficiency without anaemia. The classification can be made, for example, by the haemoglobin value and the value for the transferrin saturation (%). Reference values for haemoglobin, determined by flow cytometry or the photometric cyanohaemoglobin method, and reference values for iron, ferritin and transferrin are listed, for example, in the reference bank of the charity Institut für Laboratoriumsmedizin und Pathobiochemie (http://www.charite.de/ilp/routine/parameter.html) and in Thomas, L., Labor und Diagnose, TH Book Verlagsgesellschaft, Frankfurt/Main 1998. Transferrin saturation is as a rule >16% in patients without iron deficiency. The normal values are given in Table III which follows below.

According to M. Wick, W. Pinggera, P. Lehmann, Eisenstoffwechsel—Diagnostik und Therapien der Anämien, 4th exp. ed., Springer Verlag Vienna 1998, all forms of iron deficiency can be detected by clinical chemistry. In this context, a reduced ferritin concentration is in general accompanied by an increased transferrin in compensation and a lower transferrin saturation.

Chronic inflammatory bowel disease (IBD) is understood as meaning a chronic inflammation of the digestive tract, in particular Crohn's disease and colitis ulcerosa.

Iron(III) complex compounds with carbohydrates which can be used according to the invention preferably include those in which carbohydrates are chosen from the group consisting of dextrans and derivatives thereof, dextrins and derivatives thereof as well as pullulan, oligomers and/or derivatives thereof. The derivatives mentioned include, in particular, the hydrogenated derivatives. Iron(III) complex compounds with dextrins or oxidation products thereof are particularly preferred. Examples of the preparation of the iron(III) complex compounds according to the invention are to be found, for example, in the abovementioned patent specifications DE 14679800, WO 04037865 A1, U.S. Pat. No. 3,076,798, WO 03/087164 and WO 02/46241, the disclosure content of which, in particular in respect of the preparation processes, is to be included here in its full scope. The term “dextrins”, which are preferably used according to the invention, is a collective name for various lower and higher polymers of D-glucose units which are formed on incomplete hydrolysis of starch. Dextrins can furthermore be prepared by polymerization of sugars (e.g. WO 02083739 A2, US 20030044513 A1, U.S. Pat. No. 3,766,165). Dextrins include maltodextrins and polymaltoses, which are prepared by enzymatic cleavage of, for example, maize starch or potato starch with alpha-amylase and which are characterized by the degree of hydrolysis, expressed by the DE value (dextrose equivalent). According to the invention, polymaltose can also be obtained by acid hydrolysis of starches, in particular dextrins. The preparation of the iron(III) complex compounds which can be used according to the invention is in general carried out by reaction of iron(II) or -(III) salts, in particular iron(III) chloride, with the dextrins, in particular polymaltose, or oxidation products of the dextrins in aqueous alkaline solution (pH>7) and subsequent working up. The preparation is also achieved in a weakly acid pH range. However, alkaline pH values of, for example, >10 are preferred.

The pH is preferably increased slowly or gradually, and this can be effected, for example, by first adding a weak base, for example up to a pH of about 3; further neutralization can then be carried out with a stronger base. Possible weak bases are, for example, alkali metal or alkaline earth metal carbonates or bicarbonates, such as sodium and potassium carbonate or bicarbonate, or ammonia. Strong bases are, for example, alkali metal or alkaline earth metal hydroxides, such as sodium, potassium, calcium or magnesium hydroxide.

The reaction can be promoted by heating. For example, temperatures of the order of 15° C. up to the boiling temperature can be used. It is preferable to increase the temperature gradually. Thus, for example, the mixture can be first heated to about 15 to 70° C. and the temperature can be gradually increased up to the boiling point.

The reaction times are, for example, of the order of 15 minutes to several hours, e.g. 20 minutes to 4 hours, for example 25 to 70 minutes, e.g. 30 to 60 minutes.

When the reaction has taken place, the solution obtained can be cooled, for example, to room temperature and optionally diluted and optionally filtered. After the cooling, the pH can be adjusted to the neural point or slightly below this, for example to values of 5 to 7, by addition of acid or base. Bases which can be used are, for example, those mentioned above for the reaction. Acids include, for example, hydrochloric acid and sulfuric acid. The solutions obtained are purified and can be used directly for the preparation of medicaments. However, it is also possible to isolate the iron(III) complexes from the solution, for example by precipitation with an alcohol, such as an alkanol, for example ethanol. The isolation can also be carried out by spray drying. The purification can be carried out in the conventional manner, in particular for removal of salts. This can be carried out e.g. by reverse osmosis, it being possible for such a reverse osmosis to be carried out e.g. before the spray drying or before the direct use in medicaments.

The iron(III) complexes obtained have, for example, an iron content of 10 to 40% wt./wt., in particular 20 to 35% wt./wt. They are in general readily water-soluble. Neutral aqueous solutions having an iron content of, for example, 1% wt./vol. to 20% wt./vol. can be prepared therefrom. These solutions can be sterilized by means of heat.

Reference may be made to U.S. Pat. No. 3,076,798 in respect of the preparation of iron(III)-polymaltose complex compounds.

In a preferred embodiment of the invention, an iron(III) hydroxide-polymaltose complex compound is used. This iron(III)-polymaltose complex compound preferably has a molecular weight in the range from 20,000 to 500,000, and in a preferred embodiment 30,000 to 80,000 Dalton (determined by means of gel permeation chromatography, for example as described by Geisser et al. in Arzneim. Forsch/Drug Res. 42(11), 12.1439-1452 (1992), paragraph 2.2.5.). A particularly preferred iron(III) hydroxide-polymaltose complex compound is the commercially obtainable Maltofer® from Vifor AG, Switzerland. In a further preferred embodiment, an iron(III) complex compound with an oxidation product of one or more maltodextrins is used. This is obtainable, for example, from an aqueous iron(III) salt solution and an aqueous solution of the product of the oxidation of one or more maltodextrins with an aqueous hypochlorite solution at a pH in the alkaline range, wherein if one maltodextrin is employed the dextrose equivalent thereof is 5 to 37 and if a mixture of several maltodextrins is employed the dextrose equivalent of the mixture is 5 to 37 and the dextrose equivalent of the individual maltodextrins involved in the mixture is 2 to 40. The weight-average molecular weight Mw of the complexes obtained in this way is, for example, 30 kDa to 500 kDa, preferably 80 to 350 kDa, particularly preferably up to 300 kDa (determined by means of gel permeation chromatography, for example as described by Geisser et al. in Arzneim. Forsch/Drug Res. 42(11), 12.1439-1452 (1992), paragraph 2.2.5.). Reference may be made, for example, to WO 2004037865 A1 in this respect, the disclosure content of which is to be included in its full scope in the present Application.

Reference may be made to WO 03/087164 in respect of the preparation of iron complex compounds with hydrogenated dextrins.

Reference may be made to WO 02/46241 in respect of the preparation of iron(III)-pullulan complex compounds.

The iron(III) hydroxide complex compounds used according to the invention are preferably administered orally. In principle, however, they can also be administered parenterally, such as intravenously, and also intramuscularly. The oral daily dose is, for example, between 10 and 500 mg iron/day of use. The dose can be taken by the patient without question over a period of several months until the iron status has improved, which is reflected by the haemoglobin value, the transferrin saturation and the ferritin value. The oral administration is preferably in the form of a tablet, a capsule, an aqueous solution or emulsion, as granules, a capsule, a gel or as a sachet. The use of solutions or emulsions is particularly preferred for children, in the form of syrups or juices, drops, etc. For this, the iron(III) hydroxide-dextrin complex compounds can be brought into the suitable administration form with conventional pharmaceutical carrier or auxiliary substances. Conventional binders or lubricants, diluents, disintegrating agents, etc. can be used for this.

The use according to the invention can be effected on children, adolescents and adults suffering from chronic inflammatory bowel diseases, preferably on adults.

The use according to the invention proceeds in particular by means of improvement in the iron, haemoglobin, ferritin and transferrin values, whereby the clinical disease activity indices of the bowel condition, abdominal pain and nausea are not impaired by the treatment according to the invention.

### BRIEF DESCRIPTION OF THE FIGURE

FIG. 1 is a diagram which shows the plasma MDA levels measured in the example before and after treatment with iron(II) sulfate or iron(III)-polymaltose complex. The effect of iron(II) sulfate and iron(III)-polymaltose complex on the plasma level of malondialdehyde (MDA) in patients with chronic inflammatory bowel disease is shown. The results are expressed as the mean ±SEM. p values are given for paired comparisons.

The invention is explained and demonstrated in its mode of action by the following example.

### Example

### Patients

41 patients with chronic inflammatory bowel disease (colitis ulcerosa or Crohn's disease in the active or quiescent state) and iron deficiency (defined by the mean corpuscular volume (MCV)<80 fl or s-ferritin<15 μg/l or s-soluble transferrin receptor >1.54 mg/l) were divided into two groups in accordance with the randomization principle. Patients who had received an iron therapy or blood transfusions during the 6 weeks before the study was conducted, an azathioprine treatment starting less than two months before the start of the study or an infliximab treatment, were suffering from cobalamin or folic acid deficiency, cancer or kidney diseases or were pregnant were excluded. Analysis of the blood, urine and stool and the clinical evaluation of the disease were carried out on day 1 and 15.

### Medication

The treatment was carried out in group 1 with iron(II) sulfate (Nycoplus Ferro-Retard®, Nycomed Pharma AS, Norway) with one table (100 mg) (corresponding to 100 mg Fe²⁺) in the morning and one tablet (100 mg) in the evening between meals for 14 days, and in group 2 with iron(III)-polymaltose complex (Maltofer Filmtabletten®, Vifor International AG, Switzerland) with two tablets (200 mg in total) (corresponding to 200 mg Fe(III)) once daily in the morning during a meal for 14 days. The tablets were taken in accordance with the manufacturer's recommendations. Patient compliance was defined as the consumption of the tablets handed out, 80% being regarded as satisfactory.

### Laboratory Studies

Blood samples were taken, after fasting during the night, on the morning of day 1 and day 15.

The plasma malondialdehyde (MDA), plasma aminothiophenols, plasma vitamins A, E and C and plasma beta-carotene were determined by high-performance liquid chromatography (HPLC) as described in the literature (Svardal, A M., Manssor, M A., Ueland, P M., Anal. Biochem. 1990; 184:338-346; Vaagenes, H., Muna, Z A., Madsen, L., Berge, R K., Lipids 1998; 33:1131-1137).

Routine laboratory analyses included determination of blood haemoglobin, the blood reticulocyte count, determination of the mean corpuscular volume (MCV), the mean corpuscular haemoglobin (MCH) and the mean corpuscular haemoglobin concentration (MCHC), a blood erythrocyte count, blood leukocyte count and blood platelet count, determination of the reticulocyte haemoglobin (CHr), the hypochromic red cell count (HYPO), determination of serum ferritin and serum iron, determination of the serum total iron binding capacity, the serum soluble transferrin receptor and the serum C-reactive protein (S-CRP), measurement of the blood erythrocyte sedimentation rate (B-ESR) and determination of serum protein and serum albumin.

Urine samples were collected on the morning of day 1 and day 15 and analysed for creatinine. Butyl-hydroxy-toluene (BHT) was added to 2 ml urine to a final concentration of 20 mM. The samples were then stored at −80° C. until analysed for urine 8-isoprostaglandin F_{2α} (8-iso-PGF_{2α}). The analysis was carried out by gas chromatography/mass spectrometry in accordance with the method of Nourooz-Zadeh et al. (Nourooz-Zadeh J., Gopaul N K., Barrow S., Mallet A I., Anggard E E., J. Chromatogr. B. Biomed. Appl. 1995; 667:199-208), but was modified in respect of the urine matrix by omitting the initial hydrolysis step and using the stationary phase protocol of Lee et al. (Lee C Y., Jenner A M., Halliwell B., Biochem. Biophys. Res. Commun. 2004; 320:696-702).

### Clinical Disease Activity

The status of the clinical disease was recorded before (day 1) and after (day 15) the iron therapy. The clinical disease activity was evaluated in patients with Crohn's disease with the “Harvey-Bradshaw Simple Index of Crohn's Disease Activity” (Harvey, R F., Bradshaw, J M., Lancet, 1980; 1:514). The Harvey-Bradshaw Simple Index is based on 5 parameters: general well-being, abdominal pain, stool frequency, abdominal mass and extraintestinal complications. The maximum score is 25 and scores of ≧5 indicate active Crohn's disease.

In patients with colitis ulcerosa, the “Simple Clinical Colitis Activity Index” was recorded (Walmsley, R S., Ayres, R C., Pounder, R E., Allan, R N., Gut 1998; 43; 29-32). The Simple Clinical Colitis Activity Index is based on 6 parameters: general well-being, stool frequency during the day and during the night, urgency of defecation, blood in the stool and extraintestinal complications. The maximum score is 20 and values of ≧4 indicate active colitis ulcerosa.

The Harvey-Bradshaw Simple Index and the Simple Clinical Colitis Activity Index are the same in respect of structure and the clinical significance of a given change in the scores. In order to allow the results of patients with Crohn's disease and colitis ulcerosa to be considered together, the activity scores were calculated as the actual score divided by the maximum score.

All the patients completed the particular Crohn's Disease Activity Index (CDAI) diary card (Best, W R., Becktel, J M., Singleton, J W., Kern, F. Jr., Gastroenterology 1976; 70:439-444) in the week before the start of the iron therapy and during the two weeks of iron therapy. The CDAI diary card comprises daily recording of general well-being, abdominal pain and the number of liquid or very soft stools. The total of seven daily records gives a score for each symptom. The higher the score, the more the patient is adversely affected. The medicament was administered during the study for 14 days and the mean for the two weeks is therefore used for the analysis. The patients also documented the occurrence of nausea before and during the iron therapy.

Patients who discontinued treatment with the medicament because of a deterioration in the symptoms were included in the analysis of the clinical disease activity and the symptom scores. Their disease activity scores were increased by two points, and the symptom scores were increased by one point per day.

### Aim and Results

The primary aim of the study was a comparison of the action of oral iron(II) sulfate and oral iron(III)-polymaltose complex on markers for oxidative tissue damage. The primary results were plasma MDA and urine iso-PGF_{2α}. The second aim was comparison of the action of the two iron formulations on the clinical disease activity and specific symptoms. The treatment time was too short for a study of the clinical effectiveness on the elimination of iron deficiency.

### Statistical Analysis

The differences within and between the groups were evaluated using the paired and non-paired Student t test, and the mean of the differences and the 95% confidence interval are stated. The values were analysed using the Wilcoxon test for pair differences, and the median and range are stated. The comparison of ratios was evaluated with the Fisher Exact test. P values of less than 0.05 are considered to be statistically significant. The data were analysed using the GraphPad Prism 4 for Windows statistics software package (GraphPad Software, Inc., San Diego, USA).

### Results

41 patients (Table 1) were divided in accordance with the randomization principle for treatment either with iron(II) sulfate (n=21) or with iron(III)-polymaltose complex (n=20). 37 patients completed the study in accordance with the protocol. In these patients, counting of the tablets resulted in a comparable compliance in the patients treated with iron(II) sulfate (100% (82-100)) and with iron(III)-polymaltose complex (100% (86-100)). Three patients (1 Crohn's disease, 2 colitis ulcerosa) discontinued the intake of iron(II) sulfate after 1, 4 and days respectively, and one patient (Crohn's disease) discontinued the treatment with iron(III)-polymaltose complex after 1 day. They all suffered from intolerable bowel movements, abdominal pain and nausea. These patients were excluded from the analysis of the laboratory values, but are included in the analysis of the clinical disease activity and the symptom scores.

### Markers for Oxidative Stress

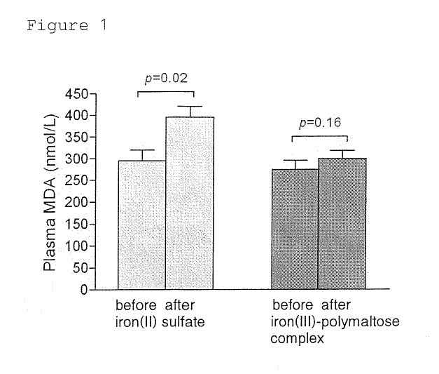

Treatment with iron(II) sulfate clearly increased the plasma MDA values by 95 nmol/l (CI 18 to 171; p=0.018) (FIG. 1) and increased the urine iso-PGF_{2α} values by 194 pg/mg creatinine (CI 58 to 447; p=0.12). Treatment with iron(III)-polymaltose complex did not significantly change the plasma MDA (p=0.16) (FIG. 1) or urine iso-PGF_{2α} (p=0.56) (Table II). The plasma vitamins A, C and E, beta-carotene, glutathione, cysteine, cysteinyl-glycine and homocysteine were unchanged after both treatments (Table II). On comparison of the treatment with iron(II) sulfate and iron(III)-polymaltose complex, the changes (before-after) in the plasma MDA (p=0.08) and urine iso-PGF_{2α} (p=0.28) do not differ significantly. However, the mean plasma MDA values of the two groups were significantly different after the particular treatment (p=0.007), higher MDA values occurring in the iron(II) sulfate group (Table II). None of the urine or plasma parameters correlated with the clinical activity index.

### Clinical Disease Activity and Symptoms

The scores of the clinical disease activity are given in Table III. Neither the treatment with iron(II) sulfate (p=0.45) nor the treatment with iron(III)-polymaltose complex (p=0.80) substantially changed the clinical disease activity indices, and the changes did not differ between the treatments (p=0.81), the number of defecations increasing during the treatment with iron(II) sulfate (from 19 (7-106) to 24 (7-55); p=0.0087), while iron(III)-polymaltose complex did not change the total number of defecations per week (from 17 (7-46) to 17 (6-66); p=0.25). Neither iron(II) sulfate nor iron(III)-polymaltose complex had an influence on the general wellbeing or on the abdominal pain score (data not given). Increased nausea was reported by 9/21 patients with iron(II) sulfate and by 7/20 patients with iron(III)-polymaltose complex (p=0.75).

### Routine Laboratory Analyses

The routine laboratory analyses are shown in Table III. Neither iron(II) sulfate nor iron(III)-polymaltose complex increased the blood haemoglobin. Only iron(II) sulfate had a significant influence on the biochemical markers of iron deficiency, with an increase in the reticulocyte haemoglobin (1.9 pg with CI 0.01 to 3.8; p=0.049), s-ferritin (12 μg/l with CI 6 to 17; p=0.0003) and blood reticulocyte count (0.016×10¹²/l with CI −0.004 to 0.036; p=0.10), and a decrease in the hypochromic red cells (−2.5% with CI −4.6 to −0.3; p=0.026), the serum soluble transferrin receptor (−0.21 mg/l with CI −0.31 to −0.11; p=0.0005) and the serum total iron binding capacity (−7 μmol/l with CI −10 to −4; p<0.0001). Iron(III)-polymaltose complex increased only the blood reticulocyte count (0.016×10¹²/l with CI 0.001 to 0.030; p 0.034).

It is clear from the results of the study that a good tolerability of the iron therapy with iron(III)-polymaltose complex is achieved in patients with chronic inflammatory bowel diseases, in particular the stool frequency being reduced compared with iron(II) sulfate and fewer patients discontinuing the study because of bowel complaints. Furthermore, the oxidative stress is significantly lower with the therapy according to the invention than with iron(II) sulfate.

TABLE I
Patient characteristics. Median (range) for age, number and
further parameters

| | Iron(II) | Iron(III)-polymaltose |
|---|---|---|
| | sulfate | complex |
| Crohn's disease/ | 13/8 | 11/9 |
| colitis ulcerosa | | |
| Female/Male | 13/8 | 12/8 |
| Age | 41 (17-69) | 31.5 (16-68) |
| Disease location | | |
| Crohn's disease* | | |
| Terminal ileum | 2 | 2 |
| Colon | 4 | 1 |
| Ileocolon | 3 | 4 |
| Upper GI | 4 | 4 |
| Disease location CU | | |
| Distal colitis | 1 | 2 |
| Subtotal colitis | 3 | 3 |
| Total colitis | 4 | 4 |
| Concurrent medication | | |
| 5-ASA | 13 | 11 |
| Sulfasalazine | 1 | 2 |
| Steroids | 7 | 5 |
| Azathioprine | 6 | 5 |
| None | 1 | 5 |

*Disease location for Crohn's disease as defined by the Vienna classification for Crohn's disease.
CU: colitis ulcerosa

TABLE II
Markers of oxidative stress. Mean (SEM).

| | | | Iron(III)-polymaltose | |
|---|---|---|---|---|
| | Iron(III) sulfate | | complex | |
| Parameter | Before | After | Before | After |
| U-8-iso-PGF_{2α} (pg/mg | 417 (46) | 629 (124) | 396 (46) | 434 (64) |
| creatinine) | | | | |
| P-malondialdehyde | 294 (25) | 395 (25)* | 275 (21) | 300 (19) |
| (nmol/l) | | | | |
| P-vitamin A (μmol/l) | 1.7 (0.2) | 1.8 (0.2) | 1.6 (0.1) | 1.9 (0.3) |
| P-vitamin C (μmol/l) | 60.9 (6.0) | 58.6 (5.4) | 61.3 (5.1) | 54.5 (5.5) |
| P-vitamin E (μmol/l) | 30.2 (1.8) | 29.3 (1.5) | 29.3 (1.6) | 28.3 (1.7) |
| P-beta-carotene | 0.67 (0.09) | 0.67 (0.10) | 0.59 (0.13) | 0.57 (0.09) |
| (μmol/l) | | | | |
| P-glutathione | 5.05 (0.48) | 5.08 (0.54) | 5.22 (0.30) | 5.43 (0.43) |
| (μmol/l) | | | | |
| P-cysteine (μmol/l) | 203 (11) | 199 (13) | 211 (11) | 209 (11) |
| P-cysteinyl-glycine | 16.7 (1.1) | 16.6 (1.2) | 18.7 (0.9) | 18.5 (1.1) |
| (μmol/l) | | | | |
| P-homocysteine | 4.87 (0.59) | 4.58 (0.47) | 6.53 (1.16) | 6.04 (0.94) |
| (μmol/l) | | | | |

*Significantly different from the level before treatment (p < 0.05).
Data from 4 patients who discontinued the treatment are not contained in the table.
P: plasma;
U: urine

TABLE III
Routine laboratory analyses. Mean (SEM)

| | | | | Iron(III)-polymaltose | |
|---|---|---|---|---|---|
| | | Iron(II) sulfate | | complex | |
| Parameter | Normal | Before | After | Before | After |
| B-haemoglobin (g/dl) | f 11.6-16.0 | 13.1 (0.4) | 13.3 (0.3) | 12.5 (0.3) | 12.5 (0.3) |
| | m 13.2-16.6 | | | | |
| B-haematocrit (%) | f 36-46 | 41 (1) | 42 (1)* | 39 (1) | 40 (1) |
| | m 37-49 | | | | |
| MCV (fl) | 80-102 | 86 (1.6) | 87 (1.3)* | 84 (1.8) | 85 (1.6) |
| MCH (pg) | 27-35 | 27 (0.8) | 28 (0.8)*# | 27 (0.7) | 27 (0.7) |
| MCHC (g/dl) | 31.0-36.0 | 31.8 (0.4) | 32.0 (0.3) | 31.6 (0.3) | 31.2 (0.3) |
| Reticulocyte | >28 | 29.3 (0.8) | 31.1 (0.7)* | 29.1 (0.8) | 29.5 (0.7) |
| haemoglobin (CHr) | | | | | |
| (pg) | | | | | |
| Hypochromic red | <5 | 10.4 (3.6) | 8.8 (3.2)* | 10.3 (3.0) | 10.6 (2.8) |
| cells (HYPO) (%) | | | | | |
| B-erythrocyte count | f 3.7-5.5 | 4.8 (0.1) | 4.8 (0.1) | 4.7 (0.1) | 4.7 (0.1) |
| (10¹²/l) | m 4.0-5.8 | | | | |
| B-reticulocyte count | 0.030-0.100 | 0.068 (0.006) | 0.084 (0.007) | 0.059 (0.006) | 0.075 (0.008)* |
| (10¹²/l) | | | | | |
| B-leukocyte count | 3.5-11.0 | 6.5 (0.5) | 6.3 (0.4) | 6.9 (0.6) | 7.0 (0.7) |
| (10⁹/l) | | | | | |
| B-platelet count | 140-400 | 324 (23) | 306 (21) | 347 (18) | 343 (21) |
| (10⁹/l) | | | | | |
| S-total iron binding | 49-85 | 81 (2) | 74 (2)*# | 77 (2) | 77 (2) |
| capacity (μmol/l) | | | | | |
| S-iron (μmol/l) | 9.0-33.0 | 11.1 (2.0) | 14.2 (2.2) | 8.8 (0.8) | 8.9 (1.5) |
| S-ferritin (μg/l) | f 15-160 | 13 (2) | 25 (3)*# | 13 (2) | 13 (2) |
| | m 25-200 | | | | |
| S-soluble | 0.84-1.54 | 1.95 (0.18) | 1.77 (0.13)* | 2.08 (0.24) | 2.03 (0.21) |
| transferrin receptor | | | | | |
| (mg/l) | | | | | |
| B-ESR (mm/h) | f < 20 | 11 (2) | 10 (2) | 22 (3) | 20 (3)* |
| | m < 15 | | | | |
| S-CRP (mg/l) | <10 | 7 (2) | 6 (2) | 12 (3) | 11 (2) |

*Significantly different from the level before treatment (p < 0.05).
#Significantly different change compared with iron(III)-polymaltose complex (p < 0.05). Data from four patients who discontinued the treatment are not contained in the table.
f: female
m: male
b: blood
s: serum

1: A method of treating iron deficiency states in patients with chronic inflammatory bowel disease comprising administering to a patient a medication comprising an iron(III) carbohydrate compound.

2: The method of claim 1, wherein the carbohydrates are selected from the group consisting of dextrans, hydrogenated dextrans, dextrins, hydrogenated dextrins, oxidized dextrins, pullulan, oligomers of pullulan, and hydrogenated pullulan.

3: The method of claim 1, wherein the carbohydrates are chosen from oxidized dextrins or hydrogenated dextrins.

4: The method of claim 1, wherein the iron(III) complex compound is an iron(III)-polymaltose complex compound.

5: The method of claim 4, wherein the iron(III)-polymaltose complex compound has a molecular weight in the range from 20,000 to 500,000.

6: The method of claim 1, wherein the iron (III) complex compound is an iron (III) complex compound with an oxidation product of one or more maltodextrins.

7: The method of claim 6, wherein the iron(III) complex compound is a water-soluble iron-carbohydrate complex obtainable from an aqueous iron(III) salt solution and an aqueous solution of the product of the oxidation of one or more maltodextrins with an aqueous hypochlorite solution at a pH in the alkaline range, and wherein if one maltodextrin is employed the dextrose equivalent thereof is 5 to 37 and if a mixture of several maltodextrins is employed the dextrose equivalent of the mixture is 5 to 37 and the dextrose equivalent of the individual maltodextrins involved in the mixture is 2 to 40.

8: The method of claim 1, wherein the medication is present in the form of a tablet, an aqueous solution or emulsion, as granules, a capsule, a gel or as a sachet.

9: The method of claim 1, wherein the chronic inflammatory bowel disease is selected from the group consisting of Crohn's disease and colitis ulcerosa.

10: The method of claim 2, wherein the carbohydrates are chosen from oxidized dextrins or hydrogenated dextrins.

11: The method of claim 2, wherein the iron(III) complex compound is an iron(III)-polymaltose complex compound.

12: The method of claim 2, wherein the iron (III) complex compound is an iron (III) complex compound with an oxidation product of one or more maltodextrins.

13: The method of claim 3, wherein the iron (III) complex compound is an iron (III) complex compound with an oxidation product of one or more maltodextrins.

14: The method of claim 4, wherein the iron (III) complex compound is an iron (III) complex compound with an oxidation product of one or more maltodextrins.

15: The method of claim 2, wherein the medicament medication is present in the form of a tablet, an aqueous solution or emulsion, as granules, a capsule, a gel or as a sachet.

16: The method of claim 3, wherein the medicament medication is present in the form of a tablet, an aqueous solution or emulsion, as granules, a capsule, a gel or as a sachet.

17: The method of claim 4, wherein the medicament medication is present in the form of a tablet, an aqueous solution or emulsion, as granules, a capsule, a gel or as a sachet.

18: The method of claim 1, wherein the chronic inflammatory bowel disease is selected from the group consisting of Crohn's disease and/or and colitis ulcerosa.

19: The method of claim 2, wherein the chronic inflammatory bowel disease is selected from the group consisting of Crohn's disease and/or and colitis ulcerosa.

20: The method of claim 3, wherein the chronic inflammatory bowel disease is selected from the group consisting of Crohn's disease and/or and colitis ulcerosa.
